(19) United States
(12) Patent Application Publication
(10) Pub. No.: US 2011/0206738 A1
(43) Pub. Date: Aug. 25, 2011

(54) FELINE STIMULANT AND METHOD OF MANUFACTURE

(76) Inventor: Valerie Loew, Fullerton, CA (US)

(21) Appl. No.: 12/712,007

(22) Filed: Feb. 24, 2010

(51) Int. Cl. A61K 9/00; A61K 36/00

(52) U.S. Cl. 424/400; 424/777

(57) ABSTRACT

According to various embodiments of the invention, stimulants are provided that result in euphoric behavior in certain animals, such as felines. Specifically, according to one embodiment, the animal stimulant is obtained by a process comprising: growing an actinidia polygama to fruition; increasing a concentration of matatabilactones within fruit of the actinidia polygama; extracting moisture from the fruit; and grounding the fruit into a fine powder. In some embodiments, the animal stimulant is specially tailored for use with feline animals.

### FIELD OF THE INVENTION

The present invention relates to feline stimulants, and more particularly, some embodiments relate to recreational substances that stimulate euphoric behavior in felines and methods of manufacturing the same.

### DESCRIPTION OF THE RELATED ART

Nepeta cataria, also known as catnip, is a substance commonly used to induce a feeling of happiness and euphoria in a majority of felines. Under the influence of catnip, cats have been observed to be attracted to the catnip, roll around the ground, and drool.

Actinidia polygama, a plant unrelated to catnip, is known to cause a similar but stronger behavioral response in felines than does catnip. Specifically, both domestic cats and large cats. such as lions, cougars, bobcats and ocelots, have been observed exhibiting large euphoric responses to actinidia polygama. In Asia, it is known to use actinidia polygama as a cat treat and even a human wellness product. However, until now, the parts of the actinidia polygama that have been used have been limited to stems, leaves, and fruit left untouched by insects.

### BRIEF SUMMARY OF EMBODIMENTS OF THE INVENTION

According to various embodiments of the invention, animal stimulants based on actinidia polygama are provided that result in a strong euphoric response in certain animals, such as felines. Specifically, according to one embodiment, the animal stimulant is obtained by a process comprising: growing an actinidia polygama to fruition; increasing concentration of matatabilactones within fruit of the actinidia polygama; extracting moisture from the fruit; and grounding the fruit into a fine powder. In some embodiments, the animal stimulant is specially tailored for use with feline animals. In addition, for some embodiments, the extraction of moisture from the fruit leaves the fruit with a moisture content of 10% to 12%.

Depending on the embodiment, increasing concentration of matatabilactones may be facilitated by exposing the fruit of the actinidia polygama to an asphondylia matatabi (commonly referred to as gall midges or gall gnats) that attack the fruit, thereby causing matatabilcatones in the fruit to increase in concentration. Additionally, in some embodiments, extracting moisture from the fruit may entail kiln drying the fruit. Once dried, in further embodiments, the fruit can be grounded into a fine powder of 80 mesh.

In additional embodiments, the fine powder may be coated on an animal toy or feral cat bait. In further embodiments, the fine powder may be developed into a salve, typically used for veterinary applications, or an animal dietary supplement.

Other embodiments include methods for manufacturing animal stimulants in accordance with various operations provided above.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present invention, in accordance with one or more various embodiments, is described in detail with reference to the following figure. The drawing is provided for purposes of illustration only and merely depict typical or example embodiments of the invention. This drawing is provided to facilitate the reader's understanding of the invention and shall not be considered limiting of the breadth, scope, or applicability of the invention.

FIG. 1 is flowchart illustrating an example method in accordance with one embodiment of the invention.

This figure is not intended to be exhaustive or to limit the invention to the precise faun disclosed. It should be understood that the invention can be practiced with modification and alteration, and that the invention be limited only by the claims and the equivalents thereof.

### DETAILED DESCRIPTION OF THE EMBODIMENTS OF THE INVENTION

The present invention is directed toward a feline stimulant and methods of manufacturing the same. In particular, the feline stimulant in accordance with embodiments of the invention is derived from the actinidia polygama plant, and results in a strong behavioral response from felines. Behavioral responses for some embodiments include the feelings of euphoria and relaxation. The types of felines susceptible to the stimulants effect include both domestic cats and larger cats, such as lions, cougars, bobcats and ocelots.

Stimulants created in accordance with the present invention can be utilized in a variety of applications including, but not limited to, coatings used for animal toys or animal bait (e.g., feral cat bait), and veterinary uses, such as dietary wellness products. For example, a stimulant in accordance with the present invention may be developed into a salve that can be applied to the fur of a sick feline. Once applied, the sick feline can then lick off the salve from its fur, thereby receiving a dose of the stimulant and causing the sick feline to have an increased appetite.

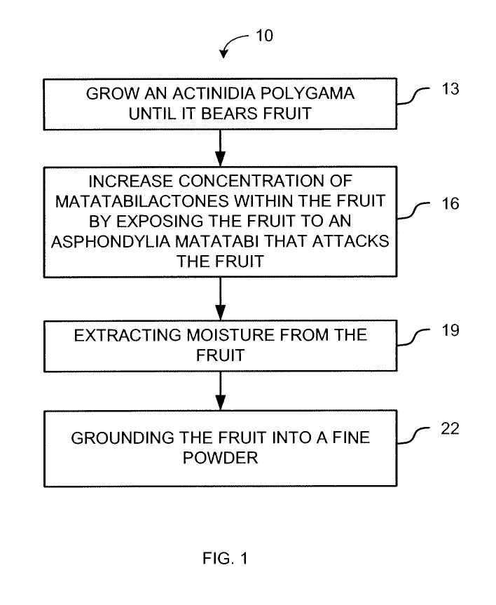

FIG. 1 provides a flowchart of an example method 10 for obtaining animal stimulant in accordance with one embodiment of the invention. Referring now to FIG. 1, method 10 begins at operation 13 with the growth of an actinidia polygama until it bears fruit. The fruit of an actinidia polygama (also known as silver vine) is a large berry. Next, during operation 16, the fruit of the actinidia polygama is exposed to at least one fly from the species of asphondylia matatabi. During this exposure, the fruit is attacked by the asphondylia matatabi (also known as a gall midge or gall gnat), thereby resulting in an increase of matatabilactones within the fruit. One of ordinary skill in the art would understand and appreciate that other methods exist for increasing the matatabilactones within the fruit produced by the actinidia polygama, and that such method are within the scope of the present invention.

Subsequently, after the fruit has been gathered from the actinidia polygama, in operation 19, moisture is extracted from the fruit in order to dry the fruit to a predetermined moisture concentration. For example, in some embodiments, the predetermined moisture concentration is between 10% and 12%. Methods for extracting moisture from the fruit include kiln drying. One of ordinary skill in the art would understand and appreciate that other conventional methods for extracting moisture can be utilized to achieve the predetermined moisture concentration without departing from the scope of the present invention.

After the fruit has been adequately dried, the fruit is ground into a fine powder in operation 22. In some embodiments, a powder of 80 mesh is utilized. In further embodiments, powder of having a finer mesh (e.g., 120 mesh, 220 mesh, etc.) may be employed. Once in powder form, the stimulant is ready to be utilized in a variety of applications such as coatings used for animal toys or animal bait (e.g., feral cat bait), as well as veterinary uses such as dietary wellness products and salves.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. An animal stimulant obtained by a process comprising:
growing an actinidia polygama to fruition;
increasing a concentration of matatabilactones within fruit of the actinidia polygama;
extracting moisture from the fruit; and
grounding the fruit into a fine powder.

2. The animal stimulant of claim 1, wherein increasing the concentration of matatabilactones comprises exposing the fruit of the actinidia polygama to an asphondylia matatabi fly that attacks the fruit, thereby causing matatabilcatones in the fruit to increase in concentration.

3. The animal stimulant of claim 1, wherein extracting moisture from the fruit causes the fruit to have a moisture content of 10% to 12%.

4. The animal stimulant of claim 1, wherein extracting moisture from the fruit comprises kiln drying the fruit.

5. The animal stimulant of claim 1, wherein the fine powder is 80 mesh powder.

6. The animal stimulant of claim 1, wherein the fine powder is finer than 80 mesh powder.

7. The animal stimulant of claim 1, wherein the animal stimulant is tailored to stimulate a feline.

8. The animal stimulant of claim 1, wherein the fine powder is coated on an animal toy or feral cat bait.

9. The animal stimulant of claim 1, wherein the fine powder is developed into a salve.

10. The animal stimulant of claim 1, wherein the fine powder is developed into an animal dietary supplement.

11. A method for creating an animal stimulant, comprising:
growing an actinidia polygama to fruition;
increasing a concentration of matatabilactones within fruit of the actinidia polygama;
extracting moisture from the fruit; and
grounding the fruit into a fine powder.

12. The method of claim 11, wherein increasing the concentration of matatabilactones comprises exposing the fruit of the actinidia polygama to an asphondylia matatabi fly that attacks the fruit, thereby causing matatabilcatones in the fruit to increase in concentration.

13. The method of claim 11, wherein extracting moisture from the fruit causes the fruit to have a moisture content of 10% to 12%.

14. The method of claim 11, wherein extracting moisture from the fruit comprises kiln drying the fruit.

15. The method of claim 11, wherein the fine powder is 80 mesh powder.

16. The animal stimulant of claim 11, wherein the fine powder is finer than 80 mesh powder.

17. The method of claim 11, wherein the animal stimulant is tailored to stimulate a feline.

18. The method of claim 11, further comprising coating an animal toy with the fine powder.

19. The method of claim 11, further comprising coating feral cat bait with the fine powder.

20. The method of claim 11, further comprising developing the fine powder into a salve.

21. The method of claim 11, further comprising developing the fine powder into an animal dietary supplement.

22. An animal stimulant comprising fruit from an actinidia polygama, wherein the fruit has been attacked by an asphondylia matatabi fly, thereby increasing a matatabilactones concentration within the fruit, wherein the fruit has a water content of 10% to 12%, and wherein the fruit is ground to a powder of 80 mesh or finer.
